(19) United States
(12) Patent Application Publication
(10) Pub. No.: US 2007/0207104 A1
(43) Pub. Date: Sep. 6, 2007

(54) Antimicrobial esters

(76) Inventor: Edward T. Borish, Westerville, OH (US)

Correspondence Address: Mitchell P. Novick, Esq.;Law Offices Of Mitchell P. Novick, 66 Park Street, Montclair, NJ, 07042, US

(21) Appl. No.: 11/714,986

(22) Filed: Mar. 6, 2007

Related U.S. Application Data
(60) Provisional application No. 60/780,016, filed on Mar. 6, 2006.

(51) Int. Cl. A61K 8/00; A61K 8/18; A61Q 1/14

(52) U.S. Cl. 424/70.1; 510/130

(57) ABSTRACT

An antimicrobial composition consists essentially of a substituted polyhydric alcohol and a fatty acid ester, the composition having the structure:

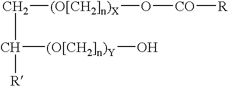

wherein R′ is H, CH₃, CH₂— (O[CH₂]n)_{z}—OH, or

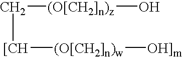

and wherein x+y, x+y+z or w+x+y+z, or combinations thereof, has an average value ranging from about 6 to about 100; R is a hydrocarbon comprising from about 3 to about 19 carbons; n is an integer ranging from about 2 to about 4 or combinations thereof; m is an integer ranging from 0 to about 6. The composition is water soluble, being characterized by an HLB value greater than 10; can be used in formulations, such as cosmetics, household and personal care products, pharmaceuticals and pet care products. Personal care products include sanitizers, lotions, shampoos, anhydrous gels, sunscreens, hair products, moisturizing products, and cleansers.

### CROSS REFERENCES TO RELATED APPLICATIONS

This application claims the benefit of U.S. Provisional Application Ser. No. 60/780,016, filed 6 Mar. 2006, the contents of which are hereby incorporated by reference herein in their entirety.

### FIELD OF THE INVENTION

Embodiments of this invention include compositions and methods using alkoxylated esters of carboxylic acids and polyhydric alcohols as antimicrobial agents and describes an improved composition and method for this purpose.

### BACKGROUND OF THE INVENTION

This invention concerns compositions and methods using certain alkoxylated esters of carboxylic acids and polyhydric alcohols as antimicrobial agents. Antimicrobial properties of certain esters are well known in the prior art. For example, U.S. Pat. No. 4,002,775 covers the use of a monoester of a polyhydric alcohol and a twelve carbon atom aliphatic fatty acid as a primary microbicide where the preferred fatty acid is the saturated straight-chain 12-carbon acid, lauric acid, and the preferred polyhydric alcohol is glycerol. It also teaches that it is difficult to predict the biological activity of long chain fatty acids and their derivatives.

Manning & Orawski teach that certain food-grade monoesters have antimicrobial properties in selected personal care emulsion formulations. They find that the preferred fatty acid is heptanoic or caprylic acid and the preferred alcohols are propylene glycol or glycerol.

Kabara (U.S. Pat. Nos. 4,067,997 and 5,208,257) teaches that ethoxylation and propoxylation tends to render certain antimicrobial agents biologically inactive. For example, nonionic ethoxylates such as Tween 80 (polyoxyethylene ((20))sorbitan monooleate) and Span 20 (sorbitan monolaurate) are not germicidally inactive, although the latter is routinely used to stop germicidal activity of chemicals. However, U.S. Patent Application Nos. US 2005/0053593 A1, 2005/0084471 A1, 2005/0058673 A1 and 2005/0089539 A1 teach that alkoxylated fatty acid esters or ethers have antimicrobial activity if alkoxylation is kept relatively low. Advantages of ethoxylation include increased water solubility and decreased irritation as the degree of ethoxylation increases. It has been disclosed that as much as 20 moles of ethylene oxide per mole of ester can be useful, but antimicrobial effectiveness decreases with increasing ethoxylation. Kabara (U.S. Pat. No. 5,208,257) discloses compositions which have been ethoxylated or propoxylated at a level of about 0.25 to 5 moles of ethoxylate or propoxylate per mole of glyceryl ester where 5 moles or less is preferred, 3 moles or less is more preferred and 1 mole or less is most preferred.

These prior art esters have relatively low polarity and correspondingly low values of hydrophile-lipophile balance (HLB), generally less than 10, and consequently have limited water solubility. Surprisingly, we find that alkoxylated esters of certain acids with polyhydric alcohols and optionally and in combination with alkoxylated polyhydric alcohols are effective antimicrobials against a wide range of microorganisms and unexpectedly that they retain that activity over a wide range of alkoxylation. For example we find that the glyceryl ester of the 8 carbon acid, ethylhexanoic acid ethoxylated with 18 moles of ethylene oxide per mole of ester (G-18-O) has activity equal to an ester that is not ethoxylated, such as glyceryl caprylate which Manning and Orawski find has exceptional activity. However, G-18-O has the advantage of being very water soluble and is totally non-irritating even when neat G-18-O is held in intimate contact with skin under occlusive conditions for 48 hours.

This is particularly surprising and unexpected since glycereth-18 ethylhexanoate has very different chemical properties than prior art compositions. Glycereth-18 ethylhexanoate has a value of HLB greater than 10, and is very water-soluble.

### BRIEF SUMMARY OF THE INVENTION

An object of the present invention is to provide an antimicrobial composition that can be used in a variety of personal care products.

An object of the present invention is to provide an antimicrobial composition that can be used as a preservative.

Another object of the present invention is to provide antimicrobial protection to the user of the composition.

Another object of the present invention is to provide a composition that can be used in the aqueous phase of multiphasic products, such as emulsions.

In one embodiment, an antimicrobial composition consists essentially of a substituted polyhydric alcohol and an ester of a fatty acid, the composition having the structure:

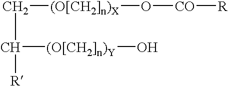

wherein R′ is H, CH₃, CH₂—(O[CH₂]n)_{z}—OH; or

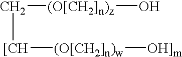

and wherein x+y, x+y+z or w+x+y+z, or combinations thereof, has an average value ranging from about 6 to about 100; wherein R is a hydrocarbon comprising from about 3 to about 19 carbons; wherein n is an integer ranging from about 2 to about 4 or combinations thereof; and wherein m is an integer ranging from 0 to about 6. The composition is water soluble, being characterized by an HLB value greater than 10; can be used in formulations, such as but not limited to cosmetics, household and personal care products, pharmaceuticals and pet care products. Personal care products include but are not limited to sanitizers, lotions, shampoos, anhydrous gels, sunscreens, hair products, moisturizing products, and cleansers.

Other embodiments of this invention utilize alkoxylated esters of carboxylic acids and polyhydric alcohols as antimicrobial agents in an improved composition and method for this purpose. The extent of alkoxylation is sufficient to increase the water solubility of the resultant material. In one embodiment of this invention, the polyhydric alcohol is glycerin and the carboxylic acid is ethylhexanoic acid. In another embodiment of this invention the composition is glycereth-18-ethylhexanoate.

### DETAILED DESCRIPTION OF THE INVENTION

This invention involves using certain alkoxylated esters of carboxylic acids and polyhydric alcohols, the extent of ethoxylation being sufficient to make the resultant material substantially water soluble. Generally these are formed by the reaction of a carboxylic acid with an excess of a polyethylene glycol ether of a polyhydric alcohol. The reaction products are complex and may be formed by other methods known to those skilled in the art. For example only, and not intended to be a limitation, ethoxylation of glyceryl carboxylic acid esters may serve the same useful function. The resultant mixture here is primarily the alkoxylated esters of carboxylic acids and polyhydric alcohols with some residual polyethylene glycol ether of a polyhydric alcohol. Alkoxylation could be done using one or more compounds chosen from the group such as, but not limited to, ethylene oxide, propylene oxide, butylene oxide and the like. Ethylene oxide and propylene oxide are employed in embodiments of the present invention. The polyhydric alcohol can be chosen from among the 2 carbon to 10 carbon polyhydric alcohols. Among them are glycerol, polyethylene glycol, propylene glycol, ribitol and sorbitol. In an embodiment of the present invention, the polyhydric alcohol is glycerol. The ester conforms generally to the formula designated as Formula I:

### Formula I:

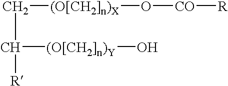

wherein R′ is H, CH₃, CH₂— (O[CH₂]ₙ)_{z}—OH; or

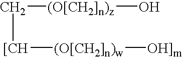

and wherein x+y, x+y+z or w+x+y+z, or combinations thereof, has an average value ranging from about 6 to about 100; wherein R is a hydrocarbon comprising from about 3 to about 19 carbons; wherein n is an integer ranging from about 2 to about 4 or combinations thereof; and wherein m is an integer ranging from 0 to about 6.

A product line designated as Hest represents various esters including alkoxylated esters of carboxylic acids and polyhydric alcohols. An exemplary material is sold under the trade name Hest G-18-O and is referred to by the INCI Name Glycereth-18 Ethylhexanoate (and) Glycereth-18. Glycereth-18 Ethylhexanoate has the molecular formula: C₄₇H₉₄O₂₂ with a molecular weight of 1010 grams per mole. Glycereth-18 Ethylhexanoate is the ester of ethylhexanoic acid with a polyethylene glycol ether of glycerin containing an average of 18 moles of ethylene oxide and conforms generally to the formula shown below (Formula 2):

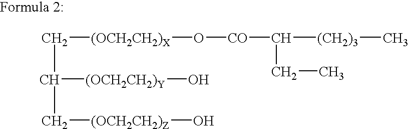

where x+y+z has an average value of 18.

The generic designation for glyceryl polyoxyethylene ethers is CAS No. 31694-55-0. Glycereth-18-ethylhexanoate is assigned CAS No. 827307-65-6, and this composition is known to include glycereth-18-hexanoate and glycereth-18; for purposes of this specification, the composition will be referred to as Glycereth-18-ethylhexanoate or G-18-0. Where the INCI name is used in the specification, the full INCI name will be stated as Glycereth-18-ethylhexanoate (and) glycereth-18. Both the term G-18-0 and the INCI name are intended to refer to the same composition.

### EXAMPLE 1

### Antimicrobial Activity of glycereth-18-hexanoate

The minimal inhibitory concentration of the composition was determined based on methods as described in the United States Pharmacopiea, National Formulary (“USP, NF”), NF 18, 1995, Microbial Assays and Journal of Applied Microbiology 89(2): 275-279 by testing microorganisms for their ability to produce visible growth on a series of agar plates containing dilutions of the composition. Mueller-Hinton is the agar medium used in these studies. Dilutions of materials to be tested were made directly into warm molten agar as described below to achieve the final concentration range (0-6%) shown in the Tables below.

Agar was prepared as recommended by the manufacturer. Sterilized agar was allowed to cool to 50° C. in a water bath and dispensing an appropriate amount into sterile containers. The agent being tested was added to the agar and thoroughly mixed. Plates were then poured and allowed to solidify at room temperature. All plates were used immediately and not stored.

Broth cultures (24 hours) were adjusted photometrically to give 107 CFU/ml (colony forming units/ml) and the plates were inoculated within 30 minutes of standardizing the inoculum. A standard inoculating loop was used to deliver and spread the inoculum on the plates. Plates were allowed to dry at room temperature before inverting and incubation. Bacterial plates were incubated at 35° C. for 48 hours while fungal plates were incubated at 25° C. for 72 hours. All plates were visually inspected immediately following incubation and the results recorded.

The organisms tested were the following: Gram-positive bacteria: Staphylococcus aureus, and Bacillus subtilis; Gram-negative bacteria: Escherichia coli and Pseudomonas aeruginosa; and Fungi: Candida albicans, Asperillus niger and Penicillium notatum.

The data below (Table 1) demonstrate the antimicrobial effectiveness of the instant invention.

TABLE 1
Minimum Inhibitory Concentration (MIC) Determinations
for Glyceryl Caprylate and Glycereth-18-Ethylhexanoate

| Concentration (%) | | | | |
|---|---|---|---|---|
| 0.15 | 0.3 | 0.6 | 1.5 | 3 |

Fungi

| | | | | | |
|---|---|---|---|---|---|
| G-18-0 | + | + | + | − | − |
| Glyceryl Caprylate | + | + | + | − | − |

Gram Positive Organisms

| | | | | | |
|---|---|---|---|---|---|
| G-18-O | + | 50% | − | − | − |
| Glyceryl Caprylate | + | 50% | − | − | − |

Gram Negative Organisms

| | | | | | |
|---|---|---|---|---|---|
| G-18-O | + | 50% | − | − | − |
| Glyceryl Caprylate | + | 50% | − | − | − |

The results demonstrate that glycereth-18-ethylhexanoate is an effective antimicrobial agent with activity against a wide range of microorganisms including Gram-positive and Gram-negative organisms as well as fungi. These results indicate that the efficacy of G-18-0 is essentially equal to that of glyceryl caprylate.

This result is noteworthy since these two agents have significantly different chemical properties. G-18-0 is substantially water-soluble while glyceryl caprylate is not, but glyceryl caprylate is substantially oil soluble. Since these materials have such different solubility properties it is surprising and unexpected that they would have such similar efficacy as antimicrobial agents.

These results mean that G-18-0 also has advantages over the prior art in finished product formulations by virtue of the water solubility of the G-18-O. Microorganisms require water to grow. For this reason, anhydrous products tend to be self-preserving. Anhydrous products are often commercialized without any added preservative. Nevertheless, preservative is often added to anhydrous products when the introduction of water is likely to occur during use. Thus, if an anhydrous product containing G-18-0 were to take up water, and the water concentration is sufficient to enable the growth of either fungi or bacteria, an antimicrobial compound would already be present in the product.

In multiphasic products, such as emulsions, microorganisms will be expected to thrive predominantly in the water phase rather than the oil phase. In these types of products, the presence of G-18-O and microorganisms would be primarily in the same phase, the water phase, while the prior art materials would not. Thus, the materials of this invention would be expected to work faster and/or at lower concentrations in these types of products. Consequently, this invention represents a noticeable improvement over the prior art.

### EXAMPLE 2

### Effect of Polyhydric Alcohol Structure, Carboxylic Acid Structure, and the Extent of Alkoxylation on Antimicrobial Activity

The effects of the structure of the polyhydric alcohol, the structure of the carboxylic acid and the extent of alkoxylation on antimicrobial activity were determined using the MIC assay as described in Example 1. These results are summarized in Table 2 below. The results below are expressed as the concentration (%) of agent corresponding to the MIC for each group of organisms tested.

TABLE 2
Minimum Inhibitory Concentration (MIC) Determinations for
Various Compounds

| | MICROORGANISMS | | |
|---|---|---|---|
| | | | Gram Positive Bacteria |
| | Fungi | Gram Negative | Staphylococcus |
| | Asperillus | Bacteria | aureus, |
| | niger, | Escherichia coli, | Staphlyococcus |
| | Candida | Pseudomonas | epidermidis, Bacillus |
| COMPOUND | albicans | aeruginosa | subtilis |
| Glycereth-18 | >=6 | >=6 | >=6 |
| Glycereth-7 | 1.5 | 3 | 3 |
| Ethylhexanoate | | | |
| Glycereth-20 | 3 | 3 | 3 |
| Ethylhexanoate | | | |
| Glycereth-18 | 1.5 | 0.6 | 0.6 |
| Ethylhexanoate* | | | |
| Glycereth-18 | >=6 | 3 | 3 |
| Benzoate | | | |
| Glycereth-18 | 1.5 | >=6 | >=6 |
| Pelargonate | | | |
| Glycereth-18 | | | |
| Laurate | >=6 | 0.6 | 0.6 |
| Propylene | | | |
| Glycol-18 | | | |
| Ethylhexanoate | 1.5 | >=6 | >=6 |
| Glycereth-40 | 1.5 | 3 | 3 |
| Ethylhexanoate | | | |

*From Table 1

It is noteworthy that all the variants tested had some antimicrobial activity except glycereth-18. Glycereth-18 showed no activity as an antimicrobial agent, indicating, but without wanting to be bound by any particular theory, that the ester component of the composition is what is contributing to the antimicrobial activity. Substituting propylene glycol as the polyhydric alcohol instead of glycerin resulted in a slight increase in activity against fungi and a slight decrease against bacteria.

The effect of ethoxylation had very little or no effect on activity in the range tested, from about 7 to about 40 moles of ethylene oxide per mole of ester.

The effect of changing the structure of the carboxylic acid had the most profound antimicrobial effect. Changing the carboxylic acid from ethylhexanoic acid (an 8 carbon branched acid) to pelargonic acid (a 9 carbon straight chained acid) had little effect on activity against fungi but greatly reduced activity against bacteria. By changing the structure of the carboxylic acid to lauric acid (a 12 carbon straight chained acid), the antimicrobial activity increased against bacteria while activity greatly decreased against fungi. Changing to the aromatic acid benzoic acid had little or no effect on activity against bacteria but greatly reduced activity against fungi.

### EXAMPLE 3

### Safety testing of Glycereth-18-Ethylhexanoate

The safety of G-18-O was tested using the neat (undiluted) material applied to the forearm of 51 human volunteers under occlusive conditions for 48 hours. These conditions are particularly aggressive, especially when compared with those in the prior art, where data on safety, if disclosed at all, is generally anecdotal using low concentrations of the agent. The dermal responses for G-18-O were scored by expert evaluators under clinical conditions according to the following 6-point scale:
0=No evidence of any effect;
+=Barely perceptible (Minimal, faint, uniform or spotty erythema);
1=Mild (Pink, uniform erythema covering most of the contact site);
2=Moderate (Pink-red erythema uniform in the entire contact site);
3=Marked (Bright re erythema with/without petechiae or papules); and
4=Severe (Deep red erythema with/without vesiculation or weeping).

Under these conditions G-18-O was totally non-irritating, with all panelists being scored as 0=No evidence of any effect. This indicates that G-18-O is exceptionally safe.

### Advantages

Surprisingly, it was found that Hest G-18-O performs unexpectedly well for controlling microbial growth. This result is particularly surprising and unexpected since Hest G-18-0 has very different chemical properties than the prior art. Composition G-18-0 has an HLB value of greater than 10, and is very water-soluble. The HLB value ranges from greater than about 10 to less than about 20, and in another embodiment, ranges from about 12 to about 16.

In contrast to prior art compositions, the composition of the instant invention and microorganisms would be primarily in the same phase, the water phase, while the prior art materials would not be so present. Thus, embodiments of this invention would be expected to work faster and/or at lower concentrations than those of the prior art in these types of products. Consequently, this invention represents a noticeable improvement over the prior art.

In various embodiments, the glycereth-18-ethylhexanoate can be used in concentrations ranging from about 0.001% to about 100% by weight of the composition. In other embodiments, the glycereth-18-ethylhexanoate can be used in concentrations ranging from about 0.01% to about 10% by weight of the composition, and in other embodiments, the glycereth-18-ethylhexanoate can be used in concentrations ranging from about 0.05% to about 8% by weight of the composition, and in still other embodiments, the glycereth-18-ethylhexanoate can be used in concentrations ranging from about 0.15% to about 3% by weight of the composition.

As will be described further below, embodiments of G-18-0 can be used in a number of different products and compositions. The composition of G-18-0 can be used as an antimicrobial alone or by being added to another solution, such as water. The composition can also be used in a variety of products, such as but not limited to cosmetics, household products, personal care products, pharmaceutical products for both humans and animals, and as a pet care or veterinary product. When used in either cosmetics or personal care products, the composition can be added into products chosen from but not limited to the group consisting of a sanitizer, a hand lotion, an antibacterial hand lotion, a shampoo, an anhydrous gel, a sunscreen, a hair gel, a moisturizing product, a moisturizing cream, a moisturizing mist, a moisturizing mist and mousse, a moisturizing lotion, a cold process lotion and a self foaming cleanser.

In some formulations, it may be necessary to increase the viscosity of the composition to enable it to adhere to a surface, or to improve how the composition feels to one's touch when it is being used or applied. One embodiment also utilizes a thickening agent of sufficient yield to stabilize the dispersion until use. Other ingredients such as, but not limited to, preservatives, colorants and fragrance can be added to the composition as desired.

Depending upon its' ultimate use, the pH of the G-18-0 may be adjusted initially, or, as it is more commonly done, the pH of the formulation is adjusted during the manufacturing process. For pH adjustment, any agent commonly used in the laboratory for pH adjustment, such as hydrochloric acid or sodium hydroxide, can be used. Other acids such as acetic acid, benzoic acid, formic acid, fumaric acid, lactic acid, phosphoric acid, sulfuric acid or other organic and/or inorganic acids, as known to those skilled in the art, could also be used. Other bases such as ammonium hydroxide, ethanolamine, magnesium hydroxide, sodium or potassium bicarbonate, sodium or potassium hydroxide, organic and/or inorganic bases, as known to those skilled in the art, could also be used. Depending upon the particular viscosifier employed in the compositions, other agents for adjusting pH could also be used. A propanol derivative, such as aminomethyl propanol, may be used to adjust the pH when a CARBOPOL® (registered trademark of Noveon, Inc., Cleveland, Ohio) is used as the viscosity modifying agent.

To facilitate maintaining an active agent in suspension during storage, shipment or prior to use, one or more of a number of viscosity modifying agents (“viscosifiers”) can be added to the composition. For example only, and not intended as any limitation, examples of viscosifiers that could be utilized include carageenans; cellulose compounds such as methyl cellulose, hydroxymethyl cellulose, and carboxymethyl cellulose; pectin; dextrans of various molecular weight ranges; starch; gum tragacanth; gum arabic; guar gum; acacia gum; gum karaya; silica, diatomaceous earth; and other commonly used agents known to those skilled in the art. The viscosifier is generally added to the composition in the range recommended by its manufacturer, and in an embodiment of the present invention, this generally ranges from about 0.1% to about 1.0%. One example of a viscosifier that can be used is an Acrylate/C10-30 Alkyl Acrylate Crosspolymer (CTFA-INCI nomenclature); it is a member of the class of viscosifiers sold commercially under the trade name CARBOPOL® (registered trademark of Noveon, Inc., Cleveland, Ohio). In other embodiments, the range can be from about 0.001% to about 10%, depending upon the combination of viscosifier employed, and the other agents contained within the composition, as recommended by the product manufacturers, and known to those skilled in the art.

One or more preservatives can be added to the composition. Examples of such preservatives are butylated hydroxyanisole (“BHA”), butylated hydroxytoluene (“BHT”), phenol, resorcinol; parabens such as methyl paraben, ethyl paraben, propyl paraben, butyl paraben, isopropyl paraben and isobutyl paraben and the like; 2-phenoxyethanol, 1,3-Octandiol; sodium benzoate, benzyl alcohol, or other preservatives commonly used in the industry. Other phenolic agents include 4,6-di-tert-butyl-resorcinol, 2,6-di-tert-butylphenol, 2,5-di-tert-butylphenol, 3,5-di-tert-butylphenol, 2,6-di-tert-hexylphenol, 2,6-di-tert-octylphenol and 2,6-di-tert-decylphenol. The general range for addition of preservatives is from about 0.01% to about 10% by weight of the composition. In one embodiment of the present invention, the preservative is added in the range of about 0.01% to about 1% by weight of the composition, and in another embodiment the preservative is added in the range of about 0.1% to about 0.6% by weight of the composition.

In one embodiment of the present invention, the preservative used is 1,3-Dimethylol-5,5-Dimethyl Hydantoin (CFTA-ICNI nomenclature of DMDM Hydantoin), and is sold commercially under the trade name GLYDANT® (registered trademark of Lonza, Inc., Fair Lawn, N.J.), or under the trademark MACKSTAT® DMDM (registered trademark of McIntyre Group Ltd., University Park, Ill.), and used in accordance with the manufacturer's specifications.

One or more agents to prevent or reduce irritation can be added to the composition. One example of an anti-irritant agent is polyvinylpyrrolodine.

A colorant can be added to the composition if desired. One or more colorants can be added to the composition, and without specifying any one in particular, it is to be understood that these colorants can be obtained from various commercial sources including but not limited to those list in the CTFA INCI Dictionary all of which are incorporated here by reference. The colorant can be added to the composition in a range from about 0.00001% to about 5%, as known to those skilled in the art. In one embodiment of the present invention, the colorant is used in the range of about 0.0001% to about 0.0005%.

A fragrance can be added to the composition if desired. One or more fragrances can be added to the composition, and without specifying any one in particular, it is to be understood that these fragrances can be obtained from various commercial sources, such as, for example only, International Flavors and Fragrances (New York, N.Y.). The fragrance can be added to the composition in a range from about 0.001% to about 20%, as known to those skilled in the art. Examples of fragrances that could be used include, for example only and are not intended to be limited, ammonium glycyrrhizate, amyl acetate, anisaldehyde, benzoic acid, betula alba extract, caraway fruit oil, safflower seed oil, caramel, cedarwood oil, cinnamyl acetate, citrus extracts such as from orange, grapefruit, lemon or lime, citronella, carrot, clove, eucalyptus, wintergreen, licorice, lavender, cherry, various berries or the like.

### EXAMPLE 4

### Moisturizing Hand Sanitizer

| | | Concentration by |
|---|---|---|
| INGREDIENT | INCI NAME | weight |
| Water | Water | 30.74% |
| Ultrez ® 21 | Acrylates/C10–30 Alkyl | 0.40% |
| | Acrylate Crosspolymer | |
| SD Alcohol 40-B | Alcohol Denatured (Ethyl | 63.00% |
| | Alcohol) | |
| AMP-95 | Aminoethyl Propanol | 0.36% |
| Hest G-18-0 | Glycereth-18 Ethylhexanoate | 5.50% |
| | (and) Glycereth-18 | |
| | | 100.00% |

The sanitizer composition is prepared by slowly hydrating the Ultrez®21 (registered trademark of Carbopol Corp.), followed by addition of the ethanol with mixing until the mixture is uniform. The aminoethyl propanol is then added and mixed until the mixture is uniform, followed by the addition of the Hest G-18-O, which is then mixed until the mixture is uniform.

### EXAMPLE 5

### Antibacterial Hand Lotion

| | | Concentration by |
|---|---|---|
| INGREDIENT | INCI NAME | weight |
| Water | Water | 22.65% |
| Ultrez ® 21 | Acrylates/C10–30 Alkyl | 0.80% |
| | Acrylate Crosspolymer | |
| SD Alcohol 40-B | Alcohol Denatured | 63.00% |
| | (Ethyl Alcohol) | |
| AMP-95 | Aminoethyl Propanol | 0.55% |
| Hest IS-3-O | Isosteareth-3 Ethylhexanoate | 10.00% |
| Hest G-18-0 | Glycereth-18 Ethylhexanoate | 3.00% |
| | (and) Glycereth-18 | |
| | | 100.00% |

The hand lotion composition is prepared by slowly hydrating the Ultrez®21, followed by addition of the ethanol with mixing until the mixture is uniform. The aminoethyl propanol is then added and mixed until the mixture is uniform, followed sequentially by the addition of the isosteareth-3 ethylhexanoate and Hest G-18-O, each of which is then mixed until the mixture is uniform.

### EXAMPLE 6

### Amide-Free Shampoo Formulation A

| | | Concentration by |
|---|---|---|
| INGREDIENT | INCI NAME | weight |
| Water | Water | 26.3% |
| Rhodapex ® EA-2 | Ammonium Laureth Sulfate | 50.0% |
| Rhodapon ® L-22 | Ammonium Lauryl Sulfate | 10.0% |
| Hetaine CDA | Disodium Cocoamidodiacetate | 3.0% |
| Hetoxamate 6000 | PEG-150 Distearate | 0.5% |
| | DS Special | |
| Hest G-18-0 | Glycereth-18 Ethylhexanoate | 10.00% |
| | (and) Glycereth-18 | |
| Mackstat ® DM | DMDM Hydantoin | 0.20% |
| | | 100.00% |

The shampoo composition is prepared by mixing the first three ingredients, and heating the mixture to about 70 degrees C. The Hetoxamate 6000 DS Special is added, and heating is continued to about 80 degrees C. The Hetaine CDA is then added followed by the Hest G-18-O, and the mixture maintained at about 80 degrees C. for a period ranging from about 5 to about 15 minutes. The mixture is then allowed to cool towards room temperature, followed by the addition of the Mackstat® DM at a temperature of about 40-45 degrees C., after which the mixture is allowed to cool to room temperature.

Rhodapex® and Rhodapon® are registered trademarks of Rhodia, Inc, North America (Cranbury, N.J.).

### EXAMPLE 7

### Amide-Free Shampoo Formulation B

| | | Concentration by |
|---|---|---|
| INGREDIENT | INCI NAME | weight |
| Water | Water | 24.3% |
| Rhodapex ® EA-2 | Ammonium Laureth Sulfate | 50.0% |
| Rhodapon ® L-22 | Ammonium Lauryl Sulfate | 10.0% |
| Mirataine ® BET- | Cocoamidopropyl Betaine | 5.0% |
| C 30 | | |
| Hetoxamate 6000 | PEG-150 Distearate | 0.5% |
| | DS Special | |
| Hest G-18-0 | Glycereth-18 Ethylhexanoate | 10.00% |
| | (and) Glycereth-18 | |
| Mackstat ® DMDM | DMDM Hydantoin | 0.20% |
| | | 100.00% |

The shampoo composition is prepared by mixing the first three ingredients, and heating the mixture to about 70 degrees C. The Hetoxamate 6000 DS Special is added, and heating is continued to about 80 degrees C. The Mirataine® (Registered trademark of Rhodia, Inc, North America, Cranbury, N.J.) BET-C 30 is then added followed by the Hest G-18-O, and the mixture maintained at about 80 degrees C. for a period ranging from about 5 to about 15 minutes. The mixture is then allowed to cool towards room temperature, followed by the addition of the Mackstat® DM at a temperature of about 40-45 degrees C., after which the mixture is allowed to cool to room temperature.

### EXAMPLE 8

### Anhydrous Gel

| | | Concentration |
|---|---|---|
| INGREDIENT | INCI NAME | by weight |
| Drakeol ® 10 | Mineral Oil | 70.0% |
| | Light | |
| Global 4075 | Glyceryl Isostearate (and) | 20% |
| | Caprylic/Capric Glycerides | |
| Hest G-18-0 | Glycereth-18 Ethylhexanoate | 5.00% |
| | (and) Glycereth-18 | |
| Sodium Stearate | Sodium Stearate | 5.0% |
| | | 100.00% |

The composition is prepared by mixing the Global 4075, glycereth-18 ethylhexanoate and sodium stearate, and heating the mixture until it is homogeneous. The mineral oil (Drakeol® 10, registered trademark of Penreco, Division of Pennzoil Inc., Butler, Pa.) is heated to the same temperature in a separate vessel. The mineral oil is then added to the mixture of Global 4075, Hest G-18-O, and sodium stearate, followed by cooling the resultant mixture to room temperature.

### EXAMPLE 9

### Sunscreen Gel Stick

| | | Concentration |
|---|---|---|
| INGREDIENT | INCI NAME | by weight |
| Hest 25B | C₁₂–C₁₅ Alkyl Benzoate | 65.5% |
| Escalol ® 557 | Octinoxate | 7.5% |
| Escalol ® 587 | Octisal | 5.0% |
| Corapan ® TQ | Diethylhexyl 2,6 | 4.1% |
| | Naphthalate | |
| Global 4075 | Glyceryl Isostearate (and) | 20.0% |
| | Caprylic/Capric Glycerides | |
| Hest G-18-0 | Glycereth-18 Ethylhexanoate | 5.0% |
| | (and) Glycereth-18 | |
| Sodium Stearate | Sodium Stearate | 5.0% |
| | | 100.0% |

The composition is prepared by mixing the Global 4075, Hest G-18-O, sodium stearate, and heating the mixture until it is homogeneous. The other four ingredients are heated to the same temperature in a separate vessel. The contents of the second vessel is then added to the mixture of Global 4075, glycereth-18 ethylhexanoate and sodium stearate, followed by cooling the resultant mixture to room temperature.

Corapan® is a registered trademark of International Specialty Products, Wayne, N.J. for chemical preparations for use in the manufacture of personal care products.

Escalol® is a registered trademark of International Specialty Products, Wayne, N.J. for ultraviolet absorbing compounds for use in the manufacture of skin care and hair care products.

### EXAMPLE 10

### Hair Gel

| | | Concentration |
|---|---|---|
| INGREDIENT | INCI NAME | by weight |
| Water | Water | 89.55% |
| Ultrez ® 21 | Acrylates/C10–30 Alkyl Acrylate | 1.00% |
| | Crosspolymer | |
| Fixative G-100 | AMP-Acrylates/Allyl Methacrylate | 3.50% |
| Polymer | Copolymer | |
| Hest G-18-0 | Glycereth-18 Ethylhexanoate | 5.00% |
| | (and) Glycereth-18 | |
| AMP-95 | Aminomethyl Propanol | 0.95% |
| | | 100.00% |

The gel composition is prepared by slowly hydrating the Ultrez-21 followed by addition of the Fixative G-100 Polymer with mixing until the mixture is uniform. The Hest G-18-O is then added and mixed mix slowly until uniform followed by the aminomethyl propanol which is then mixed until uniform.

### EXAMPLE 11

### Moisturizing Cream

| | | Concentration |
|---|---|---|
| INGREDIENT | INCI NAME | by weight |
| Hest P-4O | Pentaerythrityl Tetraoctanoate | 7% |
| Hest ICS | Isocetyl Stearate | 7% |
| Hest 25B | C12–15 Alkyl Benzoate | 7% |
| Hetoxol STA-2 | Steareth-2 | 5% |
| Hetoxol I-20-20 | Octyldodeceth-20 | 1% |
| Hest G-18-O | Glycereth-18 Ethylhexanoate | 1% |
| | (and) Glycereth-18 | |
| Water | Water | 72% |
| | | 100% |

The moisturizing cream emulsion composition is prepared by combining the Hest 25B, Hest ICS, Hest P-40, Hetoxol STA-2 and Hetoxol I-20-20 and heating the mixture to 80-85 degrees C. In a separate vessel, the Hest G-18-O and water are combined and heated to the same temperature. With adequate mixing, step 1 is slowly added to step 2 and mixing is continued for 10 minutes after which the mixing speed is reduced the composition is cooled to room temperature.

### EXAMPLE 12

### Moisturizing Mist & Mousse

| | | Concentration |
|---|---|---|
| INGREDIENT | INCI NAME | by weight |
| Water | Water | 88.9% |
| Hest G-18-O | Glycereth-18 Ethylhexanoate | 10.0% |
| | (and) Glycereth-18 | |
| Glycerin | Glycerin | 0.5% |
| | | 100.0% |

This moisturizing composition is prepared by combining the Water, Hest G-18-O and Glycerin and mixing till uniform. The composition is delivered using a pump spray or an Airspray® (registered trademark of Airspray International BV Corporation, Beverwijk, Netherlands) pump foamer.

### EXAMPLE 13

### Moisturizing Lotion

| | | Concentration |
|---|---|---|
| INGREDIENT | INCI NAME | by weight |
| Hest 25B | C12–15 Alkyl Benzoate | 5% |
| Hest TC | Tricaprylin | 10% |
| Hest T-3S | Trimethyloipropane tristearate | 3% |
| Hest P-4IS | Pentaerythrityl isostearate | 6% |
| HetoxolI-20-20 | Octyldodeceth-20 | 2% |
| Hetoxol STA-2 | Steareth-2 | 6% |
| Rest G-18-O | Glycereth-18 Ethylhexanoate | 5% |
| | (and) Glycereth-18 | |
| Carbopol ® | Acrylates/C10–30 alkyl | |
| Ultrez ® 21 | Acrylates crosspolymer | 0.2% |
| Triethanolamine | Triethanolamine | 0.3% |
| Water | Water | 62.5% |
| | | 100% |

The moisturizing lotion emulsion composition is prepared by combining the Hest 25B, Hest TC, Hest P-41S, Hest T-3S, Hetoxol STA-2, and Hetoxol I-20-20 and heating to 80-85 degrees C. In a separate vessel, Carbopol Ultrez®-21 is slowly hydrated followed by addition of the Hest G-18-O and the mixture is heated to the same temperature. With adequate mixing step 1 is slowly added to step 2 and mixing is continued for 10 minutes after which the mixing speed is reduced and the composition is cooled to room temperature.

### EXAMPLE 14

### Cold Process Lotion

| | | Concentration |
|---|---|---|
| INGREDIENT | INCI NAME | by weight |
| Water | Water | 78.7% |
| Ultrez ® 21 | Acrylates/C10–30 Alkyl Acrylate | 0.5% |
| | Crosspolymer | |
| Hest IS-3-O | Isosteareth-3 Ethylhexanoate | 6.0% |
| ANP-95 | Amninomethyl Propanol | 0.5% |
| DRY-FLO ® PC | Aluminum Starch Octenylsuccinate | 5.0% |
| Hest G-18-O | Glycereth-18 Ethylhexanoate | 5.0% |
| | (and) Glycereth-18 | |
| Silicone HL-88 | Dimethicone | 1.7% |
| Propylene Glycol | Propylene Glycol | 2.0% |
| Paragon III | Phenoxyethanol (and) | 0.6% |
| | DMDM Hydantoin (and) | |
| | Methyl Paraben (and) | |
| | Propyl Paraben | |
| | | 100.0% |

The composition is prepared by slowly hydrating the Ultrez®-21 followed by addition of the Hest IS-3-0. The AMP-95 then added. In a separate vessel the DRY-FLO PC is slurried into the Hest G-18-0. To the slurry is added the Silicone HL-88 followed by the Propylene Glycol. With adequate mixing the slurry is added to the mixture of other ingredients and mixed till uniform. The Paragon III is then added and mixed till uniform.

DRY-FLO® is a registered trademark of National Starch and Chemical Company, Bridgewater, N.J. for chemically treated dry powder used as a lubricant in the manufacture of skin care products.

### EXAMPLE 15

### Self Foaming Cleanser

| | | Concentration |
|---|---|---|
| INGREDIENT | INCI NAME | by weight |
| Water | Water | 82.0% |
| Mackam ® 2C | Disodium Cocoamphodiacetate | 12.0% |
| Hest G-18-O | Glycereth-18 Ethylhexanoate | 2.0% |
| | (and) Glycereth-18 | |
| Hetoxide G-26 | Glycereth-26 | 2.0% |
| Hetoxide G-7 | Glycereth-7 | 2.0% |
| | | 100.0% |

The cleanser composition is prepared by combining the Water, Mackam® 2C (a registered trademark of McIntyre Group, Ltd, University Park, Ill. for surfactants for use in the manufacture of personal care, household, institutional, industrial and commercial products), Hest G-18-O and mixing until uniform. The composition is delivered using an Airfoamer dispenser.

Products with the designation of either Hest, Hetaine, Hetoxamate, Hetoxol, Hetoxide or Global are products of Global Seven, Inc., Franklin N.J.

Therefore, although this invention has been described with a certain degree of particularity, it is to be understood that the present disclosure has been made only by way of illustration, and that numerous changes in the details of the composition and additives may be resorted to without departing from the spirit and scope of the invention.

What is claimed is:

1. A composition consisting essentially of a substituted polyhydric alcohol and an ester of a carboxylic acid, the composition having the structure of: 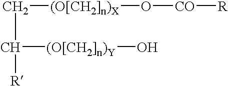
wherein R′ is H, CH₃, CH₂— (O[CH₂]ₙ)_{z}—OH, or 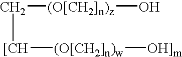
and wherein x+y, x+y+z or w+x+y+z, or combinations thereof, has an average value ranging from about 6 to about 100; wherein R is a hydrocarbon or mixture of alkyl groups comprising from about 3 to about 19 carbons; wherein n is an integer of ranging from about 2 to about 4, or combinations thereof; and m is an integer ranging from 0 to about 6.

2. The composition as described in claim 1, wherein each of w, x, y, and z has a numerical value between about 1 and about 50.

3. The composition as described in claim 1, wherein the sum of x+y, x+y+z or w+x+y+z, or combinations thereof, has an average value ranging from about 6 to about 20.

4. The composition as described in claim 1, wherein R comprises alkyl groups having carbon chain lengths ranging from C₄ to C₁₀.

5. The composition as described in claim 4, wherein R comprises alkyl groups ranging from C₆ to C₈.

6. The composition as described in claim 2, wherein the composition has the formula:

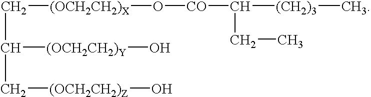

7. The composition as described in claim 2, wherein the polyhydric alcohol is selected from the group consisting of glycerol, polyethylene glycol, propylene glycol, ribitol, and sorbitol.

8. The composition as described in claim 2, wherein the polyhydric alcohol is selected from the group consisting of glycerol and polyethylene glycol.

9. The composition as described in claim 2, wherein the composition is added to a formulation, the formulation being chosen from the group consisting of a cosmetic, a household product, a personal care product, a pharmaceutical product and a veterinary product.

10. The composition as described in claim 9, wherein the personal care product is chosen from the group consisting of a sanitizer, a hand lotion, an antibacterial hand lotion, a shampoo, an anhydrous gel, a sunscreen, a hair gel, a moisturizing product, a moisturizing cream, a moisturizing mist, a moisturizing mist and mousse, a moisturizing lotion, a cold process lotion and a self foaming cleanser.

11. The composition as described in claim 9, wherein the formulation is applied to the skin or hair of a mammal.

12. The composition as described in claim 11, wherein the mammal is a human.

13. The composition as described in claim 9, wherein the composition comprises from about 0.001% to about 100% by weight of the formulation.

14. The composition as described in claim 9, wherein the composition comprises from about 0.01% to about 10% by weight of the formulation.

15. The composition as described in claim 9, wherein the composition comprises from about 0.15% to about 3% by weight of the formulation.

16. The composition as described in claim 2, wherein the composition is further characterized by having an HLB value ranging from greater than 10 to about 20.

17. The composition as described in claim 9, further comprising at least one agent selected from the group consisting of emollients, film-formers, viscosity modifiers, colorants, anti-irritants, preservatives, buffering agents, humectants and mixtures thereof.

18. The composition as described in claim 2, wherein the composition has antimicrobial activity against Staphylococcus aureus, Staphylococcus epidermidis, Bacillus subtilis, Escherichia coli and Pseudomonas aeruginosa.

19. An antimicrobial composition consisting essentially of a substituted polyhydric alcohol and an ester of a fatty acid, the composition having the structure of: 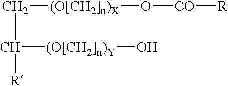
wherein R′ is H, CH₃, CH₂— (O[CH₂]ₙ)_{z}—OH, or 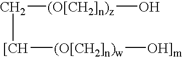
and wherein x+y, x+y+z or w+x+y+z, or combinations thereof, has an average value ranging from about 6 to about 100; wherein R is a hydrocarbon comprising from about 3 to about 19 carbons; wherein n is an integer ranging from about 2 to about 4 or combinations thereof; and wherein m is an integer ranging from 0 to about 6; the composition being present in a quantity sufficient to effectively inhibit the infectious activity of a pathogenic microorganism; and a physiologically acceptable diluent.

20. A process for inhibiting infection by a pathogenic microorganism in a subject in need thereof comprising administering to the subject an amount of a composition consisting essentially of a substituted polyhydric alcohol and an ester of a fatty acid, the composition having the structure of: 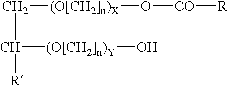
wherein R′ is H, CH₃, CH₂— (O[CH₂]ₙ)_{z}—OH, or 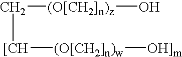
and wherein x+y, x+y+z or w+x+y+z, or combinations thereof, has an average value ranging from about 6 to about 100; wherein R is a hydrocarbon comprising from about 3 to about 19 carbons; wherein n is an integer ranging from about 2 to about 4 or combinations thereof; and wherein m is an integer ranging from 0 to about 6; the composition being administered in a concentration effective to inhibit the infectious activity of the microorganism.

21. The process as described in claim 20, further comprising the step of adding the composition to a formulation, the formulation being chosen from the group consisting of a cosmetic, a household product, a personal care product, a pharmaceutical product and a veterinary product.

22. The process as described in claim 21, wherein the personal care product is chosen from the group consisting of a sanitizer, a hand lotion, a shampoo, an anhydrous gel, a sunscreen, a hair gel, a moisturizing cream, a moisturizing mist, a moisturizing mist and mousse, a moisturizing lotion, a cold process lotion and a self foaming cleanser.

23. The process described in claim 21, comprising the step of applying the composition to the skin or hair of a mammal.

24. The process as described in claim 23, wherein the mammal is a human.

25. The process as described in claim 22, wherein the composition has antimicrobial activity against Staphylococcus aureus, Staphylococcus epidermidis, Bacillus subtilis, Escherichia coli and Pseudomonas aeruginosa.

26. The process as described in claim 22, wherein the composition has antimicrobial activity against Candida albicans, Aspergillis niger and Penicillium notatum.

27. An antimicrobial composition consisting essentially of a glycerol ester of an ethoxylated fatty acid as described in claim 2; and water.

28. The antimicrobial composition as described in claim 27, wherein the fatty acid comprises ethylhexanoate.

29. An antimicrobial composition consisting essentially of a composition having the formula: 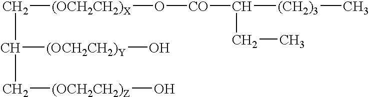
wherein x+y+z has an average value of 18.
